(19) United States
(12) Patent Application Publication
(10) Pub. No.: US 2021/0338413 A1
(43) Pub. Date: Nov. 4, 2021

(54) THERAPEUTIC DEVICES AND METHODS FOR APPLYING COMPRESSION, VIBRATION, AND/OR HEAT TO AN INDIVIDUAL

(71) Applicants: Abigale Haluska, Hobart, IN (US); Megan Jones, Hobart, IN (US); Joshua Breitsprecher, Hobart, IN (US)

(72) Inventors: Abigale Haluska, Hobart, IN (US); Megan Jones, Hobart, IN (US); Joshua Breitsprecher, Hobart, IN (US)

(21) Appl. No.: 17/245,887

(22) Filed: Apr. 30, 2021

Related U.S. Application Data
(60) Provisional application No. 63/017,756, filed on Apr. 30, 2020.

(51) Int. Cl. A61F 2/08; A61F 2/46; A61L 27/36

(52) U.S. Cl. CPC A61F 2/08; A61F 2002/30762; A61L 27/3612; A61F 2/468

(57) ABSTRACT

Therapeutic devices configured to be worn by an individual to apply compression, vibration, and/or heat to cartilage tissue at one or more joints of the individual. The individual dons the therapeutic device so that therapeutic units of the therapeutic device are located in proximity to the cartilage tissue, and electrical power is supplied to a control system and to the therapeutic units to operate and control each of the therapeutic units individually and simultaneously to selectively apply the compression, vibration, and/or heat to the cartilage tissue.

### CROSS REFERENCE TO RELATED APPLICATIONS

This application claims the benefit of U.S. Provisional Application No. 63/017,756, filed Apr. 30, 2020, the contents of which are incorporated herein by reference.

### BACKGROUND OF THE INVENTION

The present invention generally relates to therapeutic devices and methods. The invention particularly relates to therapeutic devices configured to be donned by an individual and operable to apply compression, vibration, and/or heat to cartilage tissue at one or more joints of the individual.

Arthritis is a common joint disorder that physically, mentally, and financially affects people around the world. Although there are over one hundred subcategories of arthritis, most forms of arthritis are generally associated with the reduction of and/or damage to cartilage tissue at a joint, and are characterized by inflammation of the joint, weakened blood circulation, stiffness, tenderness, and warmth. In addition to being an economic burden, arthritis may interfere with daily activities, may have a significant impact on quality of life, and may promote the progression of various other health conditions, including heart disease, diabetes, and obesity.

Severe cases of arthritis may be treated with medicines and injections. However, symptoms of less severe arthritis may be eased with heat therapy, compression, and stretching. In addition, there are many therapeutic devices commercially available that are intended to treat arthritis or symptoms thereof. For example, these devices may include various compression devices, massage devices, warming or cooling devices, and transcutaneous electrical nerve stimulation (TENS) units. Although there are numerous therapeutic devices on the market today, many of these products are heavy, bulky, and difficult to manipulate.

In view of the above, it can be appreciated that it would be desirable if therapeutic devices were available for treating arthritis or reducing its symptoms and were capable of at least partly overcoming or avoiding problems, shortcomings or disadvantages of existing therapeutic devices.

### BRIEF DESCRIPTION OF THE INVENTION

The present invention provides therapeutic devices configured to be worn by an individual and methods of operating such devices to apply compression, vibration, and/or heat to cartilage tissue at one or more joints of the individual.

According to one aspect of the invention, a therapeutic device includes a body configured to be worn by an individual, at least two therapeutic units secured to the body so as to locate the at least two therapeutic units in proximity to cartilage tissue at at least one joint of the individual, a control system configured to operate and control each of the at least two therapeutic units individually and simultaneously, and a power source configured to supply electrical power to the control system and the at least two therapeutic units. The at least two therapeutic units are operable to apply at least one of compression, vibration, and heat to the cartilage tissue while the individual is wearing the therapeutic device.

According to another aspect of the invention, a method involves an individual donning a therapeutic device so that at least two therapeutic units of the therapeutic device are located in proximity to cartilage tissue at at least one joint of the individual, providing electrical power to a control system and to the at least two therapeutic units, and operating and controlling each of the at least two therapeutic units individually and simultaneously with the control system to selectively apply compression, vibration, and/or heat to the cartilage tissue while the individual is wearing the therapeutic device.

Technical effects of the therapeutic device and method described above preferably include the capability of treating and/or reducing symptoms of arthritis or other joint-related conditions using compression, vibration, and/or heat that is selectively applied to cartilage tissue at one or more joints where symptoms of the condition exist. The therapeutic device can be constructed to have a reduced impact on activities while worn by an individual relative to certain existing therapeutic devices.

Other aspects and advantages of this invention will be appreciated from the following detailed description.

### BRIEF DESCRIPTION OF THE DRAWINGS

FIGS. 1 and 2 represent different views of a first embodiment of a therapeutic device in the form of a glove configured to be worn by an individual and to simultaneously apply compression, vibration, and/or heat to cartilage tissue at a joint of the individual in accordance with certain nonlimiting aspects of the invention.

FIG. 3 is a fragmentary view of the therapeutic device of FIGS. 1 and 2, and represents a wrist portion of glove.

FIG. 4 represents certain components of a second embodiment of a therapeutic device in the form of a glove configured to be worn by an individual and to simultaneously apply compression, vibration, and/or heat to cartilage tissue at a joint of the individual in accordance with certain nonlimiting aspects of the invention.

FIGS. 5 and 6 represent palmar and dorsal views, respectively, of a third embodiment of a therapeutic device in the form of a glove configured to be worn by an individual and to simultaneously apply vibration and/or heat to cartilage tissue at a joint of the individual in accordance with certain nonlimiting aspects of the invention.

FIG. 7 is an isolated view of a heating unit of the therapeutic device of FIGS. 5 and 6.

### DETAILED DESCRIPTION OF THE INVENTION

The intended purpose of the following detailed description of the invention and the phraseology and terminology employed therein is to describe what is shown in the drawings, which include the depiction of one or more nonlimiting embodiments of the invention, and to describe certain but not all aspects of the embodiments depicted in the drawings. The following detailed description also identifies certain but not all alternatives of the depicted embodiments. Therefore, the appended claims, and not the detailed description, describe what is believed to be one or more aspects of the invention.

Disclosed herein are therapeutic devices and methods suitable for treating and/or easing symptoms of arthritis or other tissue-related conditions. The therapeutic devices are configured to be placed in contact with and/or donned (or otherwise worn) by an individual and apply compression, vibration, and/or heat to cartilage tissue at one or more joints of the individual. Preferably, the therapeutic devices are portable, compact, and flexible such that they have minimal negative effects on daily activities of an individual using the devices. The therapeutic devices may have various structures, shapes, and components specific to certain applications. Such structures may include but are not limited to pads, wraps, sleeves, gloves, socks, and various articles of clothing. In addition to treating arthritis or its symptoms, the therapeutic devices may be used to treat other conditions and symptoms, such as carpal tunnel syndrome and general body aches and pains. The therapeutic devices may also be beneficial for treating injuries, reducing soreness after physical activities, and/or proactively reducing the likelihood of injury or soreness prior to physical activities.

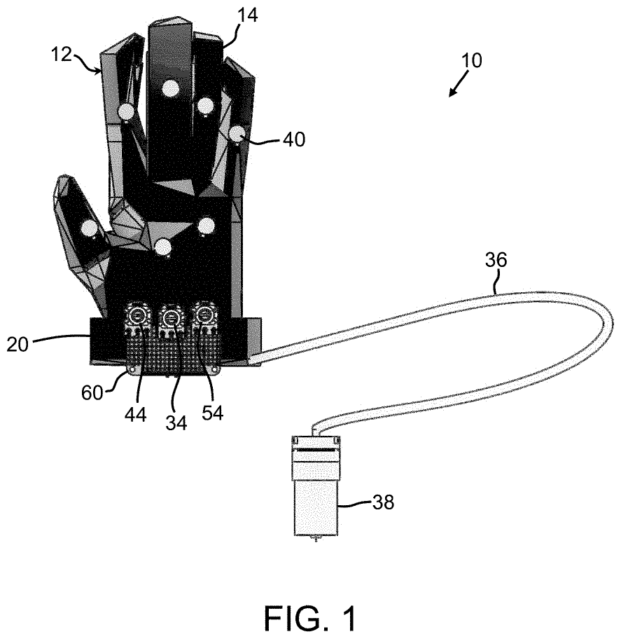

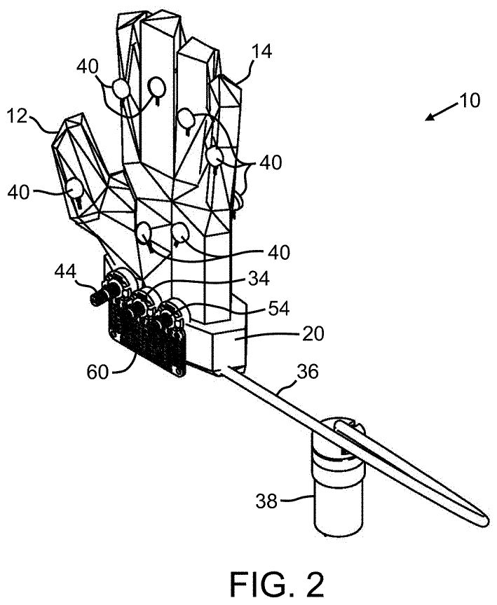

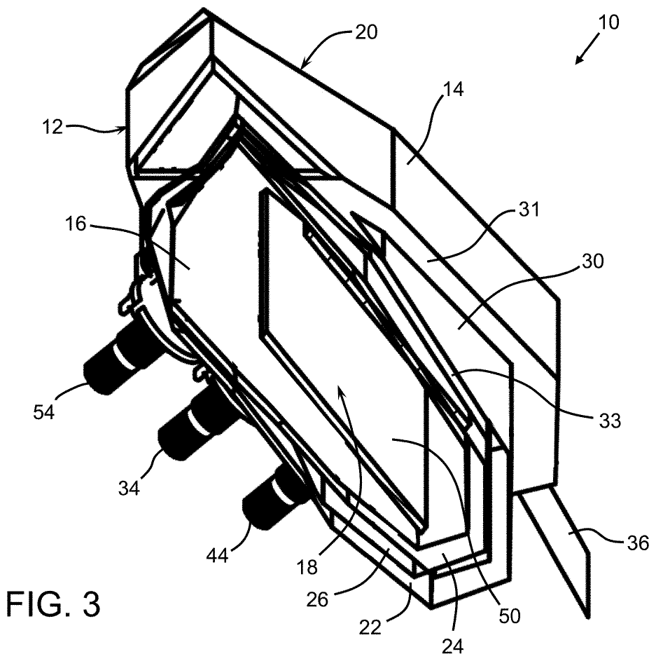

FIGS. 1 through 3 represent a first embodiment of a therapeutic device in the form of an electric therapeutic glove 10 configured to be worn by an individual to simultaneously apply compression, vibration, and/or heat to cartilage tissue at multiple joints of the individual's hand. The glove 10 includes a body 12 configured to be worn on a hand of an individual. In this embodiment, the body 12 is intended to cover an individual's hand in its entirety, though the body 12 alternatively may cover only portions of an individual's hand. The body 12 has exterior surfaces 14, interior surfaces 16, and an internal cavity 18 (FIG. 3), and may be formed of one or more layers (e.g., 22 and 24 in FIG. 3) of one or more materials, such as but not limited to natural and/or synthetic fiber materials, one or more of which may provide a thermal insulating layer between the exterior and interior surfaces 14 and 16 of the body 12 of the glove 10. As common to gloves, the body 12 includes palm and back sections, tubular finger and thumb sections, a wrist portion 20, and an opening in the wrist portion 20 that provides access to the internal cavity 18 within the body 12 that is sized and shaped to receive a hand.

In order to apply compression, vibration, heat, or a combination thereof to an individual's hand, the glove 10 includes two or more therapeutic units 30, 40, and/or 50 individually adapted to apply compression, vibration, and/or heat, respectively. As such, the therapeutic units 30, 40, and/or 50 are also referred to herein as compression units 30, vibration units 40, and heating units 50, and can be simultaneously or individually operated. The glove 10 includes a control system comprising a controller 60 for activating, deactivating, and/or modulating each of the therapeutic units 30, 40, and 50. In the embodiment represented in FIGS. 1 through 3, the control system further includes three rotatable dials or knobs 34, 44, and 54 (e.g., potentiometers) associated with the therapeutic units 30, 40, and 50, respectively. Preferably, manual rotation of the knobs 34, 44, and 54 function to activate, deactivate, and modulate the intensity (e.g., increase or decrease in compressive force applied, vibration amplitude, or temperature) of each of the corresponding therapeutic units 30, 40, and 50. It should be understood that the control system is not limited to the use of rotatable knobs as the means of controlling the therapeutic units 30, 40, and 50, nor is it limited to a single control means for each therapeutic unit 30, 40, and 50. Rather, the control system may include various quantities of various types of control means such as but not limited to electrical or mechanical buttons, switches, or touchscreen digital display interfaces, as well as control capabilities via a wireless technology signal or network. Preferably, the control system should be configured to operate each of the therapeutic units 30, 40, and 50 at an intensity and for a duration that produces a therapeutic effect to treat arthritis or another condition, or reduce one or more symptoms thereof. Such intensities and durations are known in the art and will not be discussed in any detail here.

The controller 60 is configured to operate and control the therapeutic units 30, 40, and 50 in response to the rotation of the knobs 34, 44, and 54. In addition, the control system may include other components suitable or necessary for controlling the therapeutic units 30, 40, and 50. Such components may include but are not limited to processors, circuit boards, electrical wiring, and data storage media. The glove 10 may include a battery pack (not shown) as a power source for supplying electrical power to the various systems of the glove 10, including its control system and therapeutic units 30, 40, and 50. Such a battery pack may be rechargeable, may be configured to receive disposable batteries, or both.

The glove 10 may comprise one or more compression units 30 that are part of a compression system configured to selectively apply compressive forces to one or more areas of the individual's hand while the individual is wearing the glove 10. This compression is intended to promote treatment and/or ease symptoms of various conditions by restricting or reducing swelling of the cartilage tissue and joints of the hand. The compressive forces may be implemented with various methods and components.

In the embodiment of FIGS. 1 through 3, the compression unit 30 is in the form of a pocket located between layers of the body 12, and is part of a compression system that further includes an air transport device 38 and tubing 36 that fluidically couples the compression unit 30 and the air transport device 38. FIG. 3 represents a fragmentary view of the wrist portion 20 of the glove 10 showing a portion of the internal cavity 18 of the glove 10 through the opening in the glove 10. FIG. 3 depicts the compression unit (pocket) 30 as enclosed by a relatively rigid wall 31 adjacent the exterior surfaces 14 of the body 12 and a relatively flexible wall 33 adjacent the interior surfaces 16 and the cavity 18 within the body 12. It should be understood that the location of the compression unit 30 is exemplary, and one or more compression units 30 of various shapes and sizes may be located elsewhere on the glove 10, including on various locations of the exterior surfaces 14 of the body 12, on various locations of the interior surfaces 16 of the body 12, or between layers of the body 12 in locations other than that represented in FIG. 3. Further, one or more compression units 30 may be located over some or all of the surfaces of the individual's hand when the glove 10 is worn.

Regardless of location and number, each compression unit 30 is preferably enclosed in and fluidically sealed by a membrane or layers of the body 12. For embodiments that include multiple separate compression units 30, the compression units 30 may be fluidically separate and operated independently or fluidically coupled together and operated in unison. As such, in certain embodiments the glove 10 may include a single integral compression unit 30 coupled to the air transport device 38 with a single line of the tubing 36, or include multiple separate compression units 30 each individually coupled to the air transport device 38 with one or more fluidically separate lines of the tubing 36, or include multiple separate compression units 30 coupled to the air transport device 38 in groups with one or more fluidically separate lines of the tubing 36.

In certain embodiments, the air transport device 38 is configured to supply a pressurized gaseous fluid (e.g., air) to the compression unit 30 through the tubing 36. Suitable devices for the air transport device 38 may include but are not limited to air compressors and pneumatic motors. A corresponding increase in pressure within the compression unit 30 preferably results in inflation or expansion of the pocket of the compression unit 30 in a manner that applies a compressive force on the individual's hand while wearing the glove 10. For example, the increase in pressure within the pocket may expand the compression unit 30 by pressing the flexible wall 33 in a direction away from the compression unit 30 and toward the cavity 18 of the body 12. When an individual is wearing the glove 10, expansion of the flexible wall 33 toward the cavity 18 causes a compressive force to be applied to surfaces of the individual's hand in contact with the flexible wall 33 or another layer of the glove 10 therebetween. Preferably, the air transport device 38 is adjustable such that the volume of pressurized air supplied thereby, and therefore the degree of inflation of the compression unit 30 resulting therefrom, may be selectively modulated. Such functionality provides for selective modulation of the force applied to an individual wearing the glove 10 upon inflation of the compression unit 30.

As an alternative embodiment, the compression unit 30 may comprise a foam material configured to compress upon application of a force thereto (for example, a sub-atmospheric pressure or vacuum) from an initial state having initial size, shape, and volume to a compressed state having reduced size, shape, and/or volume relative to the initial state, and to expand upon removal of the force from the compressed state to the initial state. Suitable materials for the foam material include but are not limited to various memory foam materials, such as viscoelastic polyurethane foams or low-resilience polyurethane foams that include polyurethane and other additives intended to increase the viscosity and/or density thereof. In this embodiment, the air transport device 38 is configured to remove air from within the compression unit (foam material) 30 through the tubing 36 and thereby apply a sub-atmospheric pressure (or vacuum) to the foam material preferably resulting in compression (collapsing) thereof. Suitable devices for the air transport device 38 may include but are not limited to pneumatic motors. The glove 10 is configured such that compression of the foam material results in constriction of the body 12 and application of a force in a direction toward the cavity 18 of the body 12. For example, compression of the foam material may cause the flexible wall 33 to preferentially constrict relative to the rigid wall 31, and thereby pull on layers of the body 12 such that a compressive force is applied to the individual's hand. Similar to the pressurized approach when the compression unit 30 is in the form of a pocket, the air transport device 38 is preferably adjustable with the knob 34 such that the vacuum applied thereby, and therefore the degree of compression of the foam material resulting therefrom, may be selectively modulated in order to provide for selective modulation of the force applied to an individual wearing the glove 10.

For both of the pocket and compressible foam embodiments of the compression unit 30, operation of the compression system by an individual may be substantially similar while the individual is wearing the glove 10. For example, while the air transport device 38 is inactive, the compression system does not apply a compressive force to the individual's hand. For the pressurized gas embodiment, the compression unit 30 is deflated or otherwise at an air pressure substantially equal to the surrounding atmospheric pressure. For the compressible foam embodiment, the foam material is fully expanded or otherwise in the initiate state while the air transport device 38 is inactive.

The individual may activate the air transport device 38 with the knob 34 to apply a compressive force to one or more surfaces of an individual's hand. Depending on the embodiment utilized, the compressive force may be a result of inflation of a pocket or compression of a foam material as described previously. Further, depending on the location of the compression unit 30 and the structure of the body 12, the compression system may apply compressive forces to an entirety of or only certain portions of the individual's hand. In certain embodiments, the glove 10 may be configured to allow the individual to select certain portions of the hand to receive the compressive forces. For example, this may involve an embodiment having multiple compression units 30 and applying the air transport device 38 to one or more but less than all of the compression units 30.

The glove 10 may comprise one or more vibration units 40 that are part of a vibration system configured to selectively apply vibration (i.e., oscillating forces) to one or more areas of the individual's hand while the individual is wearing the glove 10. This vibration is intended to promote treatment and/or ease symptoms of various conditions by stimulating nerves of the individual's hand. In the embodiment of FIGS. 1 through 3, the vibration system includes multiple vibration units 40 configured to produce vibration in response to application of an electric current thereto. The vibration units 40 are secured to various locations on the exterior surfaces 14 of the body 12 in positions associated with, corresponding to, adjacent to, or more preferably directly over (superimposed on) joints of the individual's hand while wearing the glove 10. In the nonlimiting embodiment of FIGS. 1 through 3, the vibration system is represented as including a total of eleven vibration units 40, of which four vibration units 40 are located on the body 12 to superimpose each of the volar (palmar) proximal interphalangeal joints, four vibration units 40 are located on the body 12 to superimpose each of the dorsal metacarpophalangeal joints of the fingers, two vibration units 40 are located on the body 12 to superimpose each of the volar (palmar) metacarpophalangeal joints of the fingers, and one vibration unit 40 is located on the body 12 to superimpose the volar (palmar) metacarpophalangeal joint of the thumb. It should be understood that the vibration units 40 may be in locations other than those represented in FIGS. 1 through 3, additional or fewer vibration units 40 may be present, and the vibration units 40 may be secured to other portions of the body 12, such as secured to interior surfaces 16 thereof or between layers thereof. Suitable devices for the vibration units 40 may include but are not limited to coin vibration motors (shaftless or pancake motors).

While wearing the glove 10, the individual may activate the vibration units 40 with the knob 44 to apply vibration to one or more surfaces of the individual's hand. Preferably, the vibration units 40 are adjustable with the knob 44 such that the intensity (i.e., amplitude) of the vibration applied thereby may be selectively modulated. Depending on the locations of the vibration units 40, the vibration system may apply vibration to an entirety of or only certain portions of the individual's hand. In certain embodiments, the glove 10 may be configured to allow the individual to select certain portions of the hand (e.g., specific joints) to receive the vibration. For example, this may involve activating one or more but less than all of the vibration units 40.

The glove 10 may comprise one or more heating units 50 that are part of a heating system configured to selectively increase the temperature at one or more areas of the individual's hand while the individual is wearing the glove 10. This temperature increase is intended to promote treatment and/or ease symptoms of various conditions by relaxing muscles and promoting lubrication of joints of the individual's hand. In the embodiment of FIGS. 1 through 3, the heating system includes a heating unit 50 configured to produce heat in response to application of an electric current thereto. As a nonlimiting example, the heating unit 50 may comprise a resistive heating element, for example, a substrate in which resistance-type heater wires are embedded and convert electrical energy into heat via Joule heating. As represented in FIG. 3, the heating unit 50 may be a pad or layer located within the cavity 18 of the body 12 and secured to one or more interior surfaces 16 of the body 12 or between layers of the body 12. Such a location may be beneficial as the body 12 or a layer thereof may act as an insulator retaining at least some of the heat produced by the heating unit 50 within the cavity 18 of the body 12. It should be understood that the location of the heating unit 50 is exemplary, and one or more heating units 50 may be located elsewhere on the glove 10, including on the exterior surfaces 14 of the body 12, on the interior surfaces 16 of the body 12 other than those represented in FIG. 3, or between layers of the body 12. Further, one or more heating units 50 may be located over some or all of the surfaces of the individual's hand when the glove 10 is worn thereby. Suitable devices for the heating unit 50 may include but are not limited to various heating materials, heating elements, and heating pads.

While wearing the glove 10, the individual may activate the heating unit 50 with the knob 54 to elevate the temperature of surfaces of the individual's hand in proximity to the heating unit 50. Preferably, the heating unit 50 is adjustable with the knob 54 such that the intensity (i.e., temperature) of the heat applied thereby may be selectively modulated. In certain embodiments, the heating unit 50 may be adjustable between a limited number of settings corresponding to predetermined temperatures. For example, the heating unit 50 may be preconfigured to be set to a predetermined low, medium, and high temperature settings. Depending on the quantity and locations of the heating unit(s) 50, the heating system may apply heat to an entirety of or only certain portions of the individual's hand. In certain embodiments, the glove 10 may be configured to allow the individual to select certain portions of the hand to receive the heat. For example, this may involve an embodiment with multiple heating unit 50 and activating one or more but less than all of the heating units 50.

In addition to the above features, the glove 10 may be configured to receive, retain, and/or couple with one or more weighted devices. For example, the glove 10 may be configured to receive and retain one or more weighted devices 26 in a pocket of the body 12, such as a pocket near or on the wrist portion 20 as represented in FIG. 3. The weighted device(s) 26 may promote treatment and/or ease symptoms of various conditions (e.g., Parkinson's disease) by weighing down the individual's hand to reduce hand tremors.

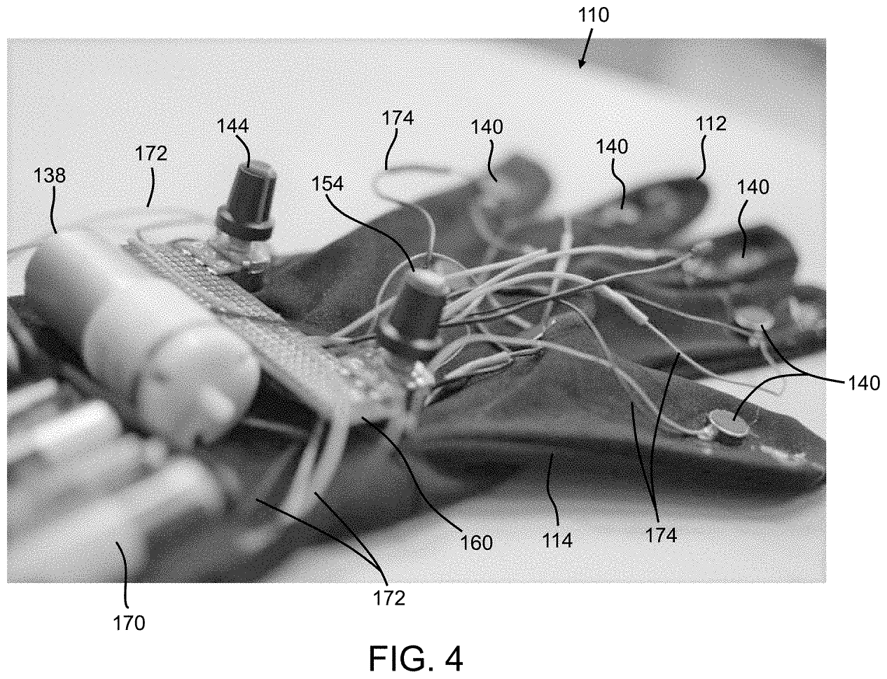

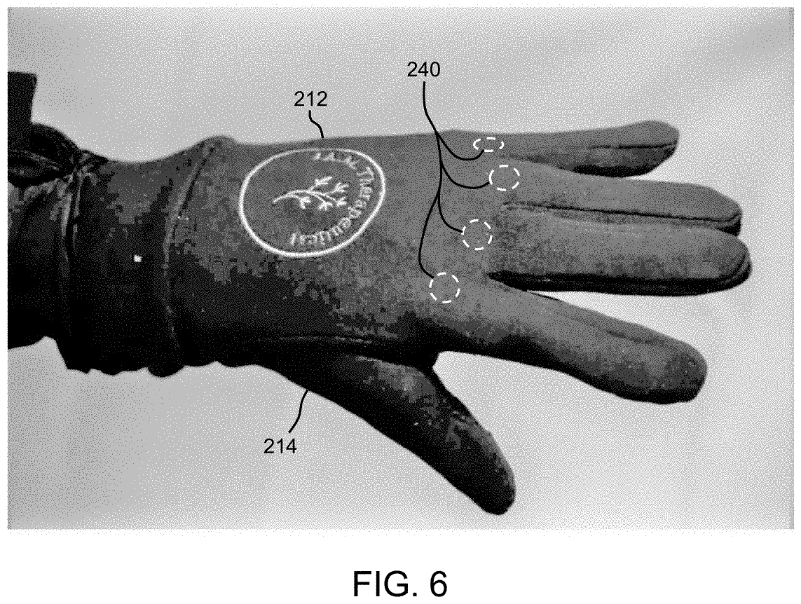

FIGS. 4 through 7 depict additional configurations of therapeutic devices in accordance with further embodiments of this invention. In these figures, consistent reference numbers are used to identify the same or functionally related/equivalent elements, but with a numerical prefix (1, 2, or 3, etc.) added to distinguish the particular embodiment from the other embodiments of the invention. In view of similarities between the embodiments, the following discussion of FIGS. 4 through 7 will focus primarily on aspects of the further embodiments that differ from the first embodiment in some notable or significant manner. Other aspects of the further embodiments not discussed in any detail can be, in terms of structure, function, materials, etc., essentially as was described for the first embodiment.

FIG. 4 represents certain components of a second embodiment of a therapeutic device in the form of an electric therapeutic glove 110 configured to be worn by an individual and apply thereto compression, vibration, or both simultaneously. The glove 110 may be used for various applications such as but not limited to treating arthritis, carpal tunnel syndrome, various other conditions, and/or symptoms thereof.

The glove 110 includes a control system comprising a controller 160 for activating, deactivating, and/or modulating compression and vibration systems carried on or within the body 112 of the glove 110. The compression and vibration systems are adapted to apply compression and vibration, and the glove 110 further includes a pair of rotatable knobs 134 and 144 for controlling the compression and vibration systems, respectively. Other than the omission of control components associated with a heating system, the control system and its controller 160 may function essentially the same as previously described for the first embodiment. A battery pack 170 is provided that is configured to provide electrical power to various systems of the glove 110. Wiring 172 electrically couples the battery pack 170 to the controller 160 to provide power thereto as well as to other components of the glove 110.

The compression system is configured to selectively apply compressive forces to one or more areas of the individual's hand while the individual is wearing the glove 110. As with the embodiment of FIGS. 1 through 3, the compression system of the glove 110 may include one or more compression units (not shown) located between layers of the body 112 of the glove 110, an air transport device 138 and tubing (not shown) fluidically coupling the compression unit(s) to the air transport device 138. The compression system and its compression units may function essentially the same as previously described for the first embodiment, and may utilize pressurized air and/or compressive foam as the compression units.

The vibration system is configured to selectively apply vibration (i.e., oscillating forces) to one or more areas of the individual's hand while the individual is wearing the glove 110. The vibration system includes a total of five vibration units 140 configured to produce a vibration in response to application of an electric current thereto. The vibration units 140 are each secured to exterior surfaces 114 of the body 112 in positions associated with the thumb and all four fingers of the hand of an individual wearing the glove 10. The vibration units 140 are electrically coupled to the controller 160 via wiring 174. The vibration system and its vibration units 140 may function essentially the same as previously described for the first embodiment.

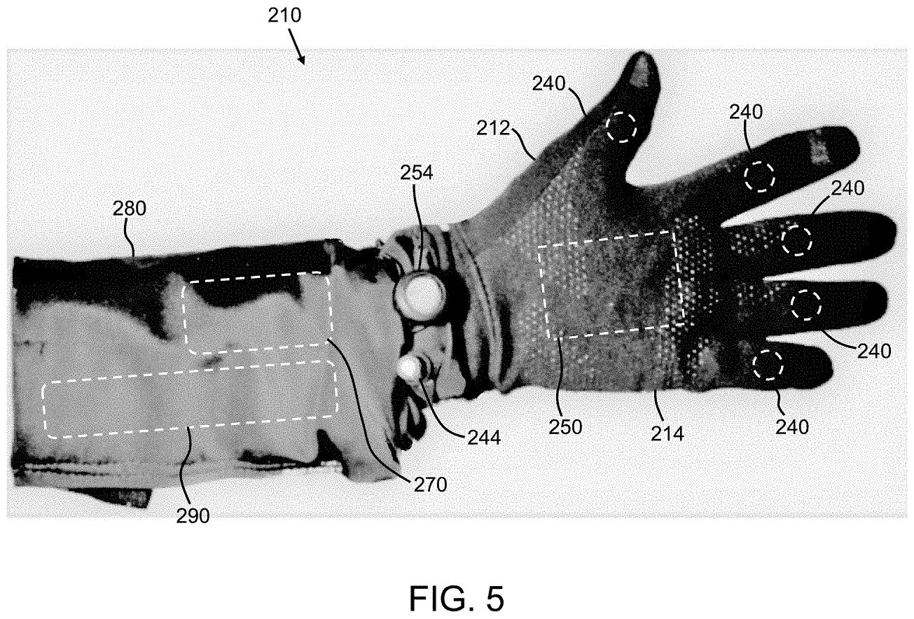

FIGS. 5 through 7 represent various views and certain components of a third embodiment of a therapeutic device in the form of an electric therapeutic glove 210 configured to be worn by an individual and apply thereto vibration, heat, or both simultaneously. The glove 210 may be used for various applications such as but not limited to treating arthritis, carpal tunnel syndrome, various other conditions, and/or symptoms thereof.

The glove 210 includes a control system comprising a controller (not shown) for activating, deactivating, and/or modulating vibration and heating systems, and includes a rotatable knob 244 and a switch 254 for controlling the vibration and heating systems, respectively. Other than the omission of control components associated a compression system, the control system of the third embodiment may function essentially the same as previously described for the first and second embodiments. A notable exception is that in this embodiment the heating system is not necessarily adjustable, but instead may be simply activated and deactivated with the switch 254 and therefore have a single predetermined temperature setting.

The vibration system is configured to selectively apply vibration (i.e., oscillating forces) to one or more areas of the individual's hand while the individual is wearing the glove 210. The vibration system includes nine vibration units 240 configured to produce a vibration in response to application of an electric current thereto. The vibration units 240 are represented as embedded in the body 212 of the glove 210 between layers of the body 212 in positions associated with the volar (palmar) proximal interphalangeal joints, the dorsal metacarpophalangeal joints of the fingers, and the volar (palmar) metacarpophalangeal joint of the thumb. The vibration system and its vibration units 240 may function essentially the same as previously described for the first and second embodiments.

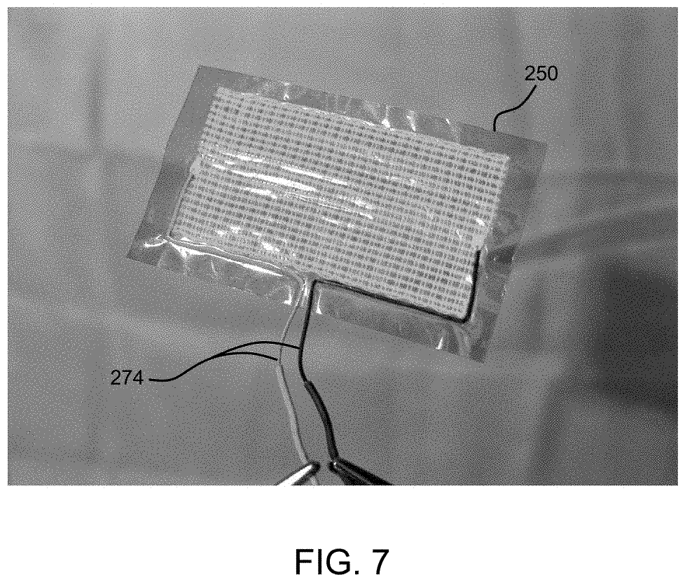

The heating system is configured to selectively increase temperatures of one or more areas of the individual's hand while the individual is wearing the glove 210. The heating system is represented as comprising a heating unit 250 embedded in the body 212 of the glove 210 and configured to produce heat in response to application of an electric current thereto. FIG. 7 is an isolated view of the heating unit 250, shown as a resistive heating element, and further shows wiring 274 for electrically coupling the heating unit 250 to the controller. Otherwise, the heating system and its heating unit 250 may function essentially the same as previously described for the first embodiment.

A sleeve 280 is provided that includes one or more internal pockets or compartments for receiving and retaining a battery pack 270 configured to provide electrical power to the various systems of the glove 210 and a weighted device 290 configured to reduce hand tremors. In certain embodiments, one or more of the finger tips of the glove 210 may be configured to allow the individual to use interactive touchscreen display devices while wearing the glove 210.

Although the glove 210 does not include a compression system, in certain embodiments the body 212 may be formed of a material of the types used for conventional compression gloves. For example, the glove 210 may be configured to be manually expanded to fit on the individual's hand, and once located thereon, the material of the glove 210 may constrict and apply a compressive force to the individual's hand.

As noted previously, the therapeutic devices may have various structures, shapes, and components specific to certain applications, including structures other than gloves. For example, such structures may further include but are not limited to pads, wraps, sleeves, socks, and various articles of clothing. The teachings disclosed herein while describing the gloves 10, 110, and 210 may be similarly applicable to therapeutic devices having any of these other structures. For example, the therapeutic devices may be configured to apply compression, vibration, and/or heat to one or more areas of an individual's arms, legs, feet, back, neck, etc. The therapeutic units 30, 40, and 50 of the therapeutic devices may apply the compression, vibration, and heat to specific anatomical areas of the individual to produce a therapeutic effect. As a nonlimiting example, a therapeutic device may provide vibration to areas adjacent to one or more of an individual's joints, such as but not limited to the individual's knees, elbows, shoulders, hips, ankles, toes, etc. These therapeutic devices may operate in substantially similar manners as described above for the gloves 10, 110, and 210.

While the invention has been described in terms of specific embodiments, it is apparent that other forms could be adopted by one skilled in the art. For example, the physical configuration of the therapeutic devices (such as the gloves 10, 110, and 210) could differ from that shown, and materials and processes/methods other than those noted could be used. In addition, the invention encompasses additional embodiments in which one or more features or aspects of different disclosed embodiments may be combined. Therefore, the scope of the invention is to be limited only by the following claims.

1. A therapeutic device comprising:
a body configured to be worn by an individual;
at least two therapeutic units secured to the body so as to locate the at least two therapeutic units in proximity to cartilage tissue at at least one joint of the individual, the at least two therapeutic units being operable to apply at least one of compression, vibration, and heat to the cartilage tissue while the individual is wearing the therapeutic device;
a control system configured to operate and control each of the at least two therapeutic units individually and simultaneously; and
a power source configured to supply electrical power to the control system and the at least two therapeutic units.

2. The therapeutic device of claim 1, wherein the at least two therapeutic units comprise at least one compression unit operable to apply compression, at least one vibration unit operable to apply vibration, and at least one heating unit operable to apply heat.

3. The therapeutic device of claim 1, wherein the body is a glove configured to be worn on a hand of the individual.

4. The therapeutic device of claim 3, further comprising one or more weighted device(s) secured to or retained by the glove to reduce tremors in the hand of the individual.

5. The therapeutic device of claim 1, wherein the at least two therapeutic units comprise vibration units operable to individually produce vibration in response to application of an electric current thereto, the vibration units being located on the body in positions so as to superimpose volar (palmar) proximal interphalangeal joints, dorsal metacarpophalangeal joints, and volar (palmar) metacarpophalangeal joint of a hand of the individual while the body is worn by the hand of the individual.

6. The therapeutic device of claim 1, wherein the at least two therapeutic units comprise at least one heating unit configured to produce heat in response to application of an electric current thereto and apply the heat to at least one area of the individual while the body is worn by the individual.

7. The therapeutic device of claim 1, wherein the at least two therapeutic units comprise a compression unit operable to apply compression, the compression unit is part of a compression system that comprises an air transport device and a tubing fluidically coupling the air transport device to the compression unit, the compression unit comprises at least one fluidically sealed pocket, and the air transport device is configured to supply a pressurized gaseous fluid to the at least one pocket causing the at least one pocket to expand and apply a compressive force to one or more areas of the individual while the body is worn by the individual.

8. The therapeutic device of claim 1, wherein the at least two therapeutic units comprise at least one compression unit operable to apply compression, the at least one compression unit being part of a compression system that comprises an air transport device and a tubing fluidically coupling the air transport device to the compression unit, the compression unit comprises a foam material, and the air transport device is configured to apply a sub-atmospheric pressure to the foam material causing the foam material to compress and apply a compressive force to one or more areas of the individual while the body is worn by the individual.

9. The therapeutic device of claim 1, wherein the control system is configured to activate, deactivate, and modulate intensities of the at least two therapeutic units.

10. The therapeutic device of claim 1, wherein the at least two therapeutic units include a compression unit operable to apply compression, a vibration unit operable to apply vibration, and a heating unit operable to apply heat, and the compression unit, the vibration unit, and the heating unit are configured to operate at intensities and for durations that produce a therapeutic effect to reduce one or more symptoms of arthritis.

11. A method comprising:
donning by an individual a therapeutic device so that at least two therapeutic units of the therapeutic device are located in proximity to cartilage tissue at least one joint of the individual;
providing electrical power to a control system and to the at least two therapeutic units; and
operating and controlling each of the at least two therapeutic units individually and simultaneously with the control system to selectively apply compression, vibration, and/or heat to the cartilage tissue while the individual is wearing the therapeutic device.

12. The method of claim 11, wherein the at least two therapeutic units comprise at least one compression unit operable to apply compression, at least one vibration unit operable to apply vibration, and at least one heating unit operable to apply heat, and the method includes operating each of the at least one compression unit, the at least one vibration unit, and the at least one heating unit individually and simultaneously with the control system to apply compression, vibration, and heat while the individual is wearing the body.

13. The method of claim 11, wherein the body is a glove and the method includes the individual wearing the glove on a hand of the individual.

14. The method of claim 13, further comprising reducing tremors in the hand of the individual with one or more weighted device(s) secured to or retained by the glove.

15. The method of claim 11, wherein the at least two therapeutic units comprise vibration units located on the body in positions that superimpose volar (palmar) proximal interphalangeal joints, dorsal metacarpophalangeal joints, and volar (palmar) metacarpophalangeal joint of a hand of the individual while the body is worn by the hand of the individual.

16. The method of claim 11, wherein the at least two therapeutic units comprise at least one heating unit, and the method includes producing heat with the at least one heating unit and applying the heat to at least one area of the individual while the body is worn by the individual.

17. The method of claim 11, wherein the at least two therapeutic units comprise a compression unit operable to apply compression, and the method includes supplying a pressurized gaseous fluid to at least one fluidically sealed pocket of the compression unit to cause the at least one pocket to expand and apply a compressive force to one or more areas of the individual while the body is worn by the individual.

18. The method of claim 11, wherein the at least two therapeutic units comprise a compression unit operable to apply compression, and the method includes applying a sub-atmospheric pressure to at least one fluidically sealed foam material to cause the at least one foam material to compress and apply a compressive force to one or more areas of the individual while the body is worn by the individual.

19. The method of claim 11, further comprising activating, deactivating, and modulating intensities of the at least two therapeutic units with the control system.

20. The method of claim 11, wherein the at least two therapeutic units include a compression unit operable to apply compression, a vibration unit operable to apply vibration, and a heating unit operable to apply heat, and the method includes operating each of the compression unit, the vibration unit, and the heating unit at intensities and for durations that produce a therapeutic effect to reduce one or more symptoms of arthritis.
